SEQUENCE LISTING (5 sequences)

SEQ ID NO: 1 - DNA, 38 bp, Artificial Sequence
  siRNA directed to human Ect2 protein of SEQ ID No.4
gcuugggaaaggcggaaugcauuccgccuuucccaagc

SEQ ID NO: 2 - DNA, 40 bp, Artificial Sequence
  siRNA directed to human Ect2 protein of SEQ ID No.4
ggacuagcuuggcagacucuagagucugccaagcuagucc

SEQ ID NO: 3 - DNA, 46 bp, Artificial Sequence
  siRNA directed to human Ect2 protein of SEQ ID No.4
gcuugggaaaggcggaaugdtdtcauuccgccuuucccaagcdtdt

SEQ ID NO: 4 - protein, 883 aa, Homo sapiens
MAENSVLTSTTGRTSLADSSIFDSKVTEISKENLLIGSTSYVEEEMPQIETRVILVQEAGKQEELIKALKDIKVGFVKMESVEEFEGLDSPEFENVFVVTDFQDSVFNDLYKADCRVIGPPVVLNCSQKGEPLPFSCRPLYCTSMMNLVLCFTGFRKKEELVRLVTLVHHMGGVIRKDFNSKVTHLVANCTQGEKFRVAVSLGTPIMKPEWIYKAWERRNEQDFYAAVDDFRNEFKVPPFQDCILSFLGFSDEEKTNMEEMTEMQGGKYLPLGDERCTHLVVEENIVKDLPFEPSKKLYVVKQEWFWGSIQMDARAGETMYLYEKANTPELKKSVSMLSLNTPNSNRKRRRLKETLAQLSRETDVSPFPPRKRPSAEHSLSIGSLLDISNTPESSINYGDTPKSCTKSSKSSTPVPSKQSARWQVAKELYQTESNYVNILATIIQLFQVPLEEEGQRGGPILAPEEIKTIFGSIPDIFDVHTKIKDDLEDLIVNWDESKSIGDIFLKYSKDLVKTYPPFVNFFEMSKETIIKCEKQKPRFHAFLKINQAKPECGRQSLVELLIRPVQRLPSVALLLNDLKKHTADENPDKSTLEKAIGSLKEVMTHINEDKRKTEAQKQIFDVVYEVDGCPANLLSSHRSLVQRVETISLGEHPCDRGEQVTLFLFNDCLEIARKRHKVIGTFRSPHGQTRPPASLKHIHLMPLSQIKKVLDIRETEDCHNAFALLVRPPTEQANVLLSFQMTSDELPKENWLKMLCRHVANTICKADAENLIYTADPESFEVNTKDMDSTLSRASRAIKKTSKKVTRAFSFSKTPKRALRRALMTSHGSVEGRSPSSNDKHVMSRLSSTSSLAGIPSPSLVSLPSFFERRSHTLSRSTTHLI

SEQ ID NO: 5 - protein, 187 aa, Homo sapiens
RWQVAKELYQTESNYVNILATIIQLFQVPLEEEGQRGGPILAPEEIKTIFGSIPDIFDVHTKIKDDLEDLIVNWDESKSIGDIFLKYSKDLVKTYPPFVNFFEMSKETIIKCEKQKPRFHAFLKINQAKPECGRQSLVELLIRPVQRLPSVALLLNDLKKHTADENPDKSTLEKAIGSLKEVMTHIN

(19) United States
(12) Patent Application Publication
(10) Pub. No.: US 2012/0231007 A1
(43) Pub. Date: Sep. 13, 2012

(54) MODULATORS OF CELL CYCLE PROGRESSION

(75) Inventors: Meng Cheong Wong, Singapore (SG); Congju Zhu, Singapore (SG); Shi Yuan Cheng, Singapore (SG)

(73) Assignee: SINGAPORE HEALTH SERVICES PTE. LTD., Singapore (SG)

(21) Appl. No.: 13/511,427

(22) PCT Filed: Nov. 23, 2010
(86) PCT No.: PCT/SG2010/000443

§ 371 Date: May 23, 2012

Related U.S. Application Data
(60) Provisional application No. 61/263,620, filed on Nov. 23, 2009.

(51) Int. Cl. A61K 39/395; A61K 31/713; C12Q 1/68; A61K 38/43; A61K 33/24; C12N 5/09; A61P 35/00

(52) U.S. Cl. 424/139.1; 435/375; 514/44 A; 424/94.1; 424/649; 435/6.12

(57) ABSTRACT

Epithelial cell transforming sequence 2 (Ect2) is a potential oncogene. Our invention uncovers key mechanisms of Ect2 oncogenicity and the development of a relevant therapies by modulation of Ect2 expression.

### INCORPORATION OF ELECTRONICALLY SUBMITTED SEQUENCE LISTING

The entirety of the sequence listing is submitted electronically at the same time as the filing of the instant application and is incorporated by reference herein.

### FIELD OF THE INVENTION

The invention relates to modulators of cell cycle progression and cell growth and methods of modulating cell cycle progression, particularly cell cycle progression from G₁ to S phase, and the use of the same particularly in treating cancer.

### BACKGROUND

The cell cycle, or cell-division cycle, is the series of events that take place in a cell leading to its division and replication. The cell cycle consists of four distinct phases. Activation of each phase is dependent on the proper progression and completion of the previous one. Cells that have temporarily or reversibly stopped dividing are said to have entered a state of quiescence. Each phase of the cell cycle has a distinct set of specialized biochemical processes that prepare the cell for initiation of cell division. Cyclin-dependent kinases (CDK) are indispensable for cell cycle progression. Antagonizing their activities is the CDK Inhibitors (CKI). To progress through G₁/S, cyclin D-CDK4/6 and cyclin E-CDK2 phosphorylates the tumour suppressor Rb and promotes E2F1-mediated transcription of S phase genes. Cyclin E-CDK2 also targets the CKI, p27^{Kip1} for degradation by phosphorylating Thr-187, facilitating recognition by the E3 ligase Skp2. p27^{Kip1} degradation is a target of Ras-medlated mitogen signalling and Ras-Induced transformation and is associated with tumour progression and poor patient prognosis. Errors within any of these processes can lead to either apoptosis or proliferative disorders such as cancer.

Cancer is one of the main diseases of current times causing 13% of all deaths globally. While there are chemicals that can affect rapidly dividing cancer cells most of these are toxic with adverse side effects. Many cancer treatments target cells which are actively undergoing cell cycle progression as the DNA is relatively exposed during cell division and hence susceptible to damage by chemicals or radiation. In general, cells are most sensitive to chemotherapy or radiation in late M and G₂ phases and most resistant in late S and late in G₁ phase. Resistance by cells during these phases results in patients' requiring prolonged and or further treatments and can contribute to ongoing oncogenesis.

G1-phase regulation includes two intertwined major themes: 1) cell growth and 2) cell cycle progression. Mammalian target of rapamycin (mTOR) pathway is a master control of cell growth. Gliomas are known to have 1) amplification/aberrant activation of receptor tyrosine kinases/Ras/MARK and PI3K/Akt/mTOR-cell growth pathways and 2) Enhanced glycolysis (also known as Warburg Effect) Both receptor tyrosine kinase activation and glucose, up-regulate mTOR activity.

Gliomas are tumors that arise from glial cells of the central nervous system mostly in the brain or spine. High-grade gliomas are highly-vascular with a tendency to infiltrate large area. As a rule, high-grade gliomas almost always grow back even after complete surgical excision. The prognosis for patients with high-grade gliomas is generally poor, and is especially so for older patients. Generaly only 50% of those diagnosed with malignant gliomas are alive 1 year after diagnosis, and 25% after two years. Those with anaplastic astrocytoma survive about three years. Glioblastoma multiforme has a worse prognosis with less than 12 month survival after diagnosis. Treatment for gliomas depends on the location, the cell type and the grade of malignancy. Often, treatment is a combined approach, using surgical resection, radiation therapy and chemotherapy. Temozolomide is a chemotherapeutic drug that is able to cross the blood-brain barrier effectively and is being used with radiation as standard care in glioma therapy. The are many cases where glioma's are reported to have developed a chemoresistance to Temozolomide. Biological or molecular targeted therapy such as single or combined inhibition of EGFR, PDGFR, VEGFR, PKC, Ras/Ra/MAPK, PI3K/Akt/mTOR, among many others has so far failed to achieve major survival advantage in glioma patients. Despite conventional therapy, median survival of malignant glioma patients remain dismal (<2 years). There is clearly a need to improve the prognosis and treatment of patients diagnosed with glioma.

Epithelial cell transforming sequence 2 (Ect2) is a member of the Db1 family of proto-oncogenes and exhibits guanine exchange activity for Rho-GTPases. It is over-expressed in rapidly dividing cells and tumours. Whilst Ect2 is implicated in oncogenesis, the mechanism is undefined. Ect2 has a domain structure similar to other Db1 family proteins. It contains a tandem Db1 homology (DH) and pleckstrin homology (PH) domain structure. Ect2 is amplified in gliomas and Ect2 expression increases with glioma tumour grade and is negatively correlated with patient survival.

While studies show that N-terminus truncation activates Ect2 as an oncogene in vitro, they do not account for the detection of only the full-length protein in tumours. Also, not all RhoGEFs are oncogenically activated by truncation. For instance, Vav1 is a RhoGEF over-expressed in several cancers as a full-length protein and there are no reports of the truncated oncogenic form. Ectopic expression of full-length Vav1 activated oncogenic signalling pathways, inducing cyclin D1 expression and cell cycle progression.).

### SUMMARY

The present invention seeks to provide novel modulators of cell cycle progression and methods of modulating cell cycle progression, particularly cell cycle progression from G₁ to S phase, and the use of the same particularly in treating or slowing cancer cells to ameliorate some of the difficulties with the current treatment of cancer. The invention further seeks to provide in vivo and in vitro methods, for arresting or slowing cell proliferation.

In one aspect the invention seeks to provide novel modulators of cell cycle progression and methods of modulating cell cycle progression and the use of the same particularly in treating or slowing glioma cells.

Accordingly the first aspect of the invention is method of modulating cell growth and cell cycle progression by controlling the concentration of epithelial cell transforming sequence 2 (Ect2) in a cellular environment.

In one embodiment when the concentration of Ect2 is increased in a cellular environment this may induce cell growth and cell cycle progression from G₁ to S phase.

In one embodiment when the concentration of Ect2 is removed, degraded or neutralised in a cellular environment this may inhibit cell growth and cell cycle progression from G₁ to S phase.

The concentration of Ect2 may be removed, degraded or neutralised by an siRNA comprising SEQ ID NO: 1 or SEQ ID NO: 2 or SEQ ID NO: 3. Alternatively, it may be removed, degraded or neutralised by an Ect2 specific antibody such as a neutralizing antibody which antibody comprises a sequence capable of binding selectively to a sequence set out in SEQ ID NO: 4 or SEQ ID NO: 5.

Another aspect of the invention provides a method for treating a patient to at least reduce a glioma growth, which comprises the step of contacting the glioma with an antagonist to epithelial cell transforming sequence 2 (Ect2).

The antagonist may comprise an siRNA comprising SEQ ID NO: 1 or SEQ ID NO: 2. Alternatively, the antagonist may comprise an Ect2 specific antibody which antibody comprises a sequence capable of binding selectively to a sequence set out in SEQ ID NO: 4 or SEQ ID NO: 5. Preferably the antibody engages the DH domain of Ect2 which antibody comprises a sequence capable of binding selectively to a sequence set out in SEQ ID NO: 5. Preferably the antibody is a neutralizing or catalytic antibody.

Another aspect of the invention provides a composition comprising a modulator of cell cycle progression capable of controlling the concentration of epithelial cell transforming sequence 2 (Ect2).

In one embodiment the modulator comprises an agonist to Ect2.

In another embodiment the modulator comprises an antagonist to Ect2. Preferably the antagonist siRNA comprising SEQ ID NO: 1 or SEQ ID NO: 2 or SEQ ID NO: 3 or an antibody to Ect2 which antibody comprises a sequence capable of binding selectively to a sequence set out in SEQ ID NO: 4 or SEQ ID NO: 5. Preferably the antibody engages the DH domain of Ect2 which antibody comprises a sequence capable of binding selectively to a sequence set out in SEQ ID NO: 5. Preferably the antibody is a neutralizing or catalytic antibody.

In one embodiment the antagonist is used in the preparation of a medicament for treating a patient with cancer, preferably glioma.

Aspects of the invention may be used together with chemotherapy. Preferably the chemotherapy agent is selected from Temozolomide; cisplatin, platinum, carboplatin; gemcitabine, paclitaxel, docetaxel, etoposide, vinorelbine, topotecan, or irinotecan; tyrosine kinase inhibitors Axitinib, Bosutinib, Cediranib, Dasatinib, Erlotinib, Gefitinib, Imatinib, Lapatinib, Lastaurtinib, Nilotinib, semaxanib, sunitinib, vandetanib, vatalanib, Wortmannin; apoptosis inducing enzymes, TNF polypeptides, TRAIL R1, TRAIL R2, Apoptosis inhibitor 2, FasL, Exisulind; molecules which hamper cell growth such as 2-Deoxy-D-glucose, oligomycin, or Rapamycin or its analogues. In one embodiment the chemotherapy agent is Temozolomide. In one embodiment the chemotherapy agent is 2-Deoxy-D-glucose. In one embodiment the chemotherapy agent is apoptosis inhibitor 2. In one embodiment the chemotherapy agent is rapamycin or its analogues.

Other aspects and advantages of the invention will become apparent to those skilled in the art from a review of the ensuing description, which proceeds with reference to the following illustrative drawings.

### BRIEF DESCRIPTION OF THE DRAWINGS

FIG. 1: Ect2 knockdown impedes cell cycle progression. A. FACS histograms showing the effect of Ect2 suppression on cell cycle entry in re-stimulated quiescent human glioma cells. B. Histogram comparison of percentage of G1 cells. Asterix denotes persistence of G1 cells in Ect2 siRNA transfected cells. Error bars indicate standard deviations. Data shown are representative of three independent experiments. C. Ect2 knockdown induces G1/S cell cycle arrest. D. Ect2 and p21 expression follows opposite trends at G1/S border.

FIG. 2: Ect2 down-regulation up-regulates p27^{Kip1} protein and impairs Rb hyper-phosphorylation. Immunoblots showing changes in p27^{Kip1} abundance and Rb phosphorylation in serum-stimulated quiescent glioma cells. Lysates were collected at the indicated time points and subjected to denaturing SDS-PAGE.

FIG. 3: Ect2 over-expression suppresses p27^{Kip1}. A. Protein lysates were collected at the indicated time points following transfection of pXJ41-Ect2 full length and analyzed using Western blotting, and immunoblotted for p27^{Kip1}, phospho-Rb, p21^{Cip1} and Ect2. Actin was used as a loading control B. U118 glioma cells were starved for 24 h before transfection with pXJ41-Ect2 full length and collected 48 h later for protein analysis.

FIG. 4: Ect2 over-expression induces serum-independent DNA synthesis. A, B. FACS histograms showing the effects on Ect2 over-expression on cell cycle progression and DNA synthesis. U118MG cells were transfected with either empty plasmid or full-length Ect2 under serum-starved or serum-supplemented conditions. Standard deviations were calculated based on 3 independent experiments. C. FACS histograms showing Ect2 induces serum-independent G1/S progression.

FIG. 5: Ect2 over-expression promotes p27^{Kip1} degradation. A. Histogram showing the effect of Ect2 over-expression on p27^{Kip1} transcript abundance. Standard deviations were calculated based on 5 sets of independent experiments (*: p<0.01, **: p<0.5) B. Histogram displaying relative luciferase activities normalized against background from empty reporter plasmid. Standard deviations were calculated from 3 independent experiments (p<0.05). C. Cells were incubated with Actinomycin D to halt transcription. p27^{Kip1} mRNA abundance was expressed as a nonlinear regression curve where the half-life (D) was derived from (n=3, p<0.05) E. immunoblot showing the effect of Ect2 over-expression and proteasome inhibition on p27^{Kip1} degradation.

FIG. 6: Ect2 activates RhoA to suppress p27^{Kip1}. Immunoblots showing the activation of RhoA by Ect2. Cells were transfected with pXJ41 plasmid expressing full length Ect2. C3 was added at 10 μg/ml 24 h later and cells were collected for protein analysis at the indicated time points. Rhotekin-binding assay was performed to pull down activated RhoA upon Ect2 over-expression. WCE: whole cell extract.

FIG. 7: Ect2 DH domain alone is sufficient to suppress p27^{Kip1}. Immunoblots showing the effect of over-expression of pXJ41-Ect2, ΔN-Ect2-DH/PH/C, ΔN-Ect2-DH/PH and ΔN-Ect2-DH on p27^{Kip1} abundance and Rb hyper-phosphorylation. Ect2 was detected with either anti-Ect2 antibody or anti-HA antibody. Actin was used as a loading control.

FIG. 8: Ect2 is found in both cytoplasmic and chromatin-bound fractions during interphase. Immunoblots showing the distribution of Ect2 in cellular compartments using low and high salt buffers. MEK was used as a cytoplasmic marker and Histone H3 as a chromatin fraction marker.

FIG. 9: Ect2 is regulated by nutrients in an mTOR-dependent manner. A. Ect2 protein levels and mTOR activity in glucose starved glioma cells are dose-dependently up-regulated by glucose (left panel). The trends are not observed with the addition of specific mTOR inhibitor, Rapamycin (right panel). B. Ect2 protein levels and mTOR activity in L-glutamine starved glioma cells are time-dependently up-regulated by L-glutamine (left panel). The trends are not observed with the addition of specific mTOR inhibitor, Rapamycin (right panel). C. U118 glioma cells were serum-starved for 72 hr then repleted with 10 ng/ml of Epithelial/Fibroblast/Insulin-like growth factors (EGF/FGF/IGF) or 10% Fetal bovine serum. ECT2 expression increased significantly at 24 hr with a corresponding increase in mTOR activity measured by p70S6 kinase phosphorylation.

FIG. 10: Ect2 is enriched in the nucleolus following serum starvation. The nucleolus is marked by B23 protein (green). Ect2 protein is detected with red fluorescence. The nucleus is stained blue with dapi.

FIG. 11: Ect2 binds to DNA and regulates ribosome biogenesis. A. Immunoblot showing the distribution of Ect2 in whole cell extract (WCE), soluble cytoplasmic fraction (S2) and chromatin-bound fraction (P3) in serum starved glioma cells following serum repletion. B. Quantitative real-time PCR showing significant down regulation (p<0.001) of pre-ribosome RNA transcription following siRNA-induced knockdown of Ect2. Serum starvation and mTOR inhibition with Rapamycin were used as controls. The insert picture shows levels of mature ribosome RNAs (lane 1-4, control, Rapamycin, -FBS, Ect2 siRNA, respectively).

FIG. 12: Ect2 regulates glioma cell growth. A. Ect2 knockdown by siRNA dose-dependently inhibits tumour cell growth rate. B. Ect2 knockdown by siRNA dose-dependently inhibits colony formation capacity of tumour cells. C. Ect2 knockdown by siRNA markedly reduces tumour cell size.

FIG. 13: Ect2 is essential for glioma cell migration. A. colony migration of tumour cells by a control vector verses siRNA Ect2 knockdown shows that siRNA Ect2 knockdown inhibits colony migration of tumour cells.

FIG. 14: Ect2 knockdown enhances TMZ chemosensitivity in glioma cells.

FIG. 15: Ect2 knockdown induces autophagy in glioma cells.

FIG. 16: Ect2 Knockdown inhibits DNA synthesis.

FIG. 17: Ect2 expression either Ect2C (top row) or Ect2F (bottom row) efficiently suppresses p21 levels. A. is Ect2, B. is p21, C. is DAPI, D. is phase contrast, and E. is a merge of the expression profiles.

FIG. 18: ECT2 regulation by RTK/mTOR pathway through EGFR inhibition with Erlotinib (10 uM) (A), Akt inhibition with API2 (10 uM), and mTOR inhibition with Rapamycin (50 nM) (B).

FIG. 19: ECT2 regulation by glucose and Warburg effect were evaluated in U118 glioma cells with glycolysis or oxidative phosphorylation inhibition using 2-Deoxy-glucose (2DG) and oligomycin respectively. Filipin III was used to permeabilise cells to provide exogenous source of pyruvate for the Tricarboxylic acid (TCA) cycle. Cellular energy level was measured by western blot analysis of AMPK phosphorylation. Cells with glycolysis inhibition showed higher level of phosphorylated AMPK, significantly lower mTOR activity and reduced ECT2 expression whereas oxidative phosphorylation inhibtion did not affect mTOR activity or ECT2 expression.

FIG. 20: Compositions of Ect2 inhibitors and chemotherapeutic agents. A. Growth inhibition induced by Ect2 knockdown or glycolysis and RTK/PI3K/Akt/mTOR inhibitors. B. Ect2 knockdown significantly enhances glycolysis and RTK/PI3K/Akt/mTOR inhibitor-induced growth inhibition. C. Ect2 knockdown further enhances combined inhibition induced by glycolysis and RTK/PI3K/Akt/mTOR inhibitors. D. Real time-PCR quantification of 45S pre-rRNA to assess the effect of ECT2 knockdown on ribosome biogenesis. U118 glioma cells showed an increase in pre-rRNA level (important pre-cursor for ribosome biogenesis) after glucose repletion whereas this increase is suppressed in cells with ECT2 knockdown.

FIG. 21: Rapamycin increases nucleolar sequestration of Ect2.

### DETAILED DESCRIPTION

The invention relates to a modulator of cell cycle progression. The modulator being capable of controlling the concentration of epithelial cell transforming sequence 2 (Ect2) in a cellular environment. The cellular environment may be an in vitro or an in vivo cellular environment. Preferably the in vivo environment is at a tumor or cancer site such as a solid tumor, a glioma or in body fluids.

The modulator may comprise either an agonist or an antagonist. An agonist increases the concentration of Ect2 thereby inducing cell cycle progression from G₁ to S phase. An antagonist inhibits, removes, degrades or neutralises the concentration of Ect2 there by inhibiting cell cycle progression from G₁ to S phase.

Inhibition of Ect2 expression in cancer cells leads to: inhibition of cancer cell growth; cancer cell cycle arrest from G1 to S phase; Suppression of cancer cell ribosome biogenesis hampering cancer cell growth; reducing invasiveness of cancer cells; sensitising cancer cells to chemotherapeutic treatment with temozolomide; and significantly improving suppression of cancer cell growth in combination with chemotherapeutic agents.

### Agonist

An agonist may comprise a full-length human Ect2 cloned into an expression vector and transfected in the cellular environment thereby increasing the amount of Ect2 protein in the cellular environment and inducing cell cycle progression. An agonist may also comprise a truncated Ect2 mutant containing the DH domain of SEQ ID No. 4 cloned into the expression vector and transfected in the cellular environment.

An agonist may be identified by screening a compound comprising the steps of: contacting a cell with the sample compound; and detecting whether the sample compound enhances cell cycle progression from G₁ to S phase in accordance with the assays listed below.

An agonist of the invention may be useful in producing cell lines. Such cell lines may be useful research tools to study cancer progression particularly glioma progression.

### Antagonist

The antagonist capable of inhibiting, removing, degrading or neutralising the concentration of Ect2 may be an siRNA. Sequences of Ect2 siRNA, which were used to knockdown Ect2 RNA and Ect2 protein to achieve relevant biological and therapeutic effect, are listed as follows:

SEQ ID NO: 1 sequence:
Sense: 5′-GCUUGGGAAAGGCGGAAUG-3′
Anti-sense: 5′-CAUUCCGCCUUUCCCAAGC-3′
SEQ ID NO: 2 sequence:
Sense: 5′-GGACUAGCUUGGCAGACUCU-3′
Anti-sense: 5′-AGAGUCUGCCAAGCUAGUCC-3′
SEQ ID NO: 3 sequence:
Sense: GCUUGGGAAAGGCGGAAUGdTdT
Antisense: CAUUCCGCCUUUCCCAAGCdTdT

Other siRNA Sequences were purchased from Ambion Inc. These siRNA sequence were directed to Ect2 and were able to knockdown the Ect2 protein but the sequence information was not released by the manufacturer. The sequences may be purchased from Ambion Inc. under catalogue numbers #16704, ID #26257 and #16704, ID #26264. SiRNA may be delivered to cell using methods known in the art such as liposome delivery or vector delivery or any other method that would be considered suitable by a person skilled in the art to deliver interfering RNA molecules.

Consistent with the invention there are provided effective antagonists of cell cycle progression from G₁ to S phase comprising (a) antibodies capable of hybridising to the full length protein sequence of Ect2 and (b) antibodies that engage the DH domain of Ect2. Exemplary antibodies include polyclonal, monoclonal, humanized, bispecific, catalytic and heteroconjugate antibodies. The antibodies of the invention are manufactured using techniques known in the art.

An exemplary full length sequence of Ect2 protein is SEQ ID NO. 4.

MAENSVLTSTTGRTSLADSSIFDSKVTEISKENLLIGSTSYVEEEMPQI
ETRVILVQEAGKQEELIKALKDIKVGFVKMESVEEFEGLDSPEFENVFV
VTDFQDSVFNDLYKADCRVIGPPVVLNCSQKGEPLPFSCRPLYCTSMMN
LVLCFTGFRKKEELVRLVTLVHHMGGVIRKDFNSKVTHLVANCTQGEKF
RVAVSLGTPIMKPEWIYKAWERRNEQDFYAAVDDFRNEFKVPPFQDCIL
SFLGFSDEEKTNMEEMTEMQGGKYLPLGDERCTHLVVEENIVKDLPFEP
SKKLYVVKQEWFWGSIQMDARAGETMYLYEKANTPELKKSVSMLSLNTP
NSNRKRRRLKETLAQLSRETDVSPFPPRKRPSAEHSLSIGSLLDISNTP
PESSINYGDTPKSCTKSSKSSTPVSKQSARWQVAKELYQTESNYVNILA
TIIQLFQVPLEEEGQRGGPILAPEEIKTIFGSIPDIFDVHTKIKDDLED
LIVNWDESKSIGDIFLKYSKDLVKTYPPFVNFPEMSKETIIKCEKQKPR
FHAFLKINQAKPECGRQSLVELLIRPVQRLPSVALLLNDLKKHTADENP
DKSTLEKAIGSLKEVMTHINEDKRKTEAQKQIFDVVYEVDGCPANLLSS
HRSLVQRVETISLGEHPCDRGEQVTLFLFNDCLEIARKRHKVIGTFRSP
HGQTRPPASLKHIHLMPLSQIKKVLDIRETEDCHNAFALLVRPPTEQAN
VLLSFQMTSDELPKENWLKMLCRHVANTICKADAENLIYTADPESFEVN
TKDMDSTLSRASRAIKKTSKKVTRAFSFSKTPKRALRRALMTSHGSVEG
RSPSSNDKHVMSRLSSTSSLAGIPSPSLVSLPSFFERRSHTLSRSTTHL
I

The DH domain of Ect2 is: SEQ ID NO. 5.

RWQVAKELYQTESNYVNILATIIQLFQVPLEEEGQRGGPILAPEEIKTI
FGSIPDIFDVHTKIKDDLEDLIVNWDESKSIGDIFLKYSKDLVKTYPPF
VNFFEMSKETIIKCEKQKPRFHAFLKINQAKPECGRQSLVELLIRPVQR
LPSVALLLNDLKKHTADENPDKSTLEKAIGSLKEVMTHIN

An antagonist to cell growth and cell cycle progression from G₁ to S phase may be identified by screening a sample compound comprising the steps of: contacting a cell culture with a sample compound; detecting the concentration of epithelial cell transforming sequence 2 (Ect2) in the cell; and detecting the concentration of Ect2 in a second cell culture not contacted with the sample compound, whereby a decrease in the Ect2 concentration within the cell culture contacted with the sample compound in relation to the second cell culture indicates the sample compound is an antagonist.

Preferably the first and second cell cultures are human glioma cells. Preferably, the antagonist inhibits cell growth and cell cycle progression from G₁ to S phase measured in accordance with the detection assays for measuring growth and progression of cell cycle as mentioned in the description below.

### Method for Treating a Patient with Cancer

On the basis of the above, the present invention provides a method for treating a patient with cancer, which comprises the step of: contacting the cells within and around a cancer with an antagonist of Ect2 capable of removing, degrading or neutralising the concentration of Ect2 within the cellular environment of the cancer. Desirably, the antagonist is provided in a therapeutic effective amount. In one embodiment the antagonist forms a compound with a chemotherapeutic agent as discussed below.

An alternative form of the present invention resides in the use of the antagonist in the manufacture of a medicament for treating a patient with cancer preferably a medicament used in treatment to affect cells over expressing Ect2.

“Treatment” and “treat” and synonyms thereof refer to both therapeutic treatment and prophylactic or preventative measures, wherein the object is to prevent or slow down (lessen) a cancer condition. Those in need of such treatment include those already diagnosed with cancer.

As used herein a “therapeutically effective amount” of a compound will be an amount of active antagonist that is capable of preventing or at least slowing down (lessening) a cancer condition, in particular increasing the average 1-1.5 year survival rate of glioma cancer patents. Dosages and administration of an antagonist of the invention in a pharmaceutical composition may be determined by one of ordinary skill in the art of clinical pharmacology or pharmacokinetics. An effective amount of the antagonist to be employed therapeutically will depend, for example, upon the therapeutic objectives, the route of administration, and the condition of the mammal. Accordingly, it will be necessary for the therapist to titer the dosage and modify the route of administration as required to obtain the optimal therapeutic effect. A typical daily dosage might range from about 10 ng/kg to up to 100 mg/kg of the mammal's body weight or more per day, preferably about 1 μg/kg/day to 10 mg/kg/day.

### Compositions of the Invention

Antagonists produced according to the invention can be administered for the treatment of cancer in the form of pharmaceutical compositions.

Thus, the present invention also relates to compositions including pharmaceutical compositions comprising a therapeutically effective amount of an antagonist that binds to Ect2 with high affinity. As used herein a compound will be therapeutically effective if it is able to affect cancer growth either in vitro or in vivo.

Pharmaceutical forms of the invention suitable for injectable use include sterile aqueous solutions (where water soluble) or dispersions and sterile powders for the extemporaneous preparation of sterile injectable solutions and or one or more carrier. Alternatively, injectable solutions may be delivered encapsulated in liposomes to assist their transport across cell membrane. Alternatively or in addition such preparations may contain constituents of self-assembling pore structures to facilitate transport across the cellular membrane. It must be stable under the conditions of manufacture and storage and must be preserved against the contaminating/destructive action of microorganisms such as, for example, bacteria and fungi.

The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as, for example, lecithin, by the maintenance of the required particle size in the case of dispersion and by the use of surfactants. Preventing the action of microorganisms in the compositions of the invention is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active antagonist in the required amount in the appropriate solvent with several of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and freeze-drying, to yield a powder of the active ingredient plus any additional desired ingredient from previously sterile-filtered solution thereof.

When the active ingredients, in particular siRNA contemplated within the scope of the invention, are suitably protected they may be orally administered, for example, with an inert diluent or with an edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active peptide in such therapeutically useful compositions in such that a suitable dosage will be obtained. Preferred compositions or preparations according to the present invention are prepared so that a dosage unit form contains between about 0.1 μg and 20 g of active compound.

The tablets, troches, pills, capsules and the like may also contain binding agents, such as, for example, gum, acacia, corn starch or gelatin. They may also contain an excipient, such as, for example, dicalcium phosphate. They may also contain a disintegrating agent such as, for example, corn starch, potato starch, alginic acid and the like. They may also contain a lubricant such as, for example, magnesium stearate. They may also contain a sweetening agent such a sucrose, lactose or saccharin. They may also contain a flavouring agent such as, for example, peppermint, oil of wintergreen, or cherry flavouring.

When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparaben as preservatives, a dye and flavouring such as, for example, cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents may also include any and all solvents, dispersion media, coatings, antibacterials and/or antifungals, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated.

Supplementary active ingredients can also be incorporated into the compositions. Preferably those supplementary active ingredients are anticancer agents such as chemotherapy agents like, for example; Temozolomide; cisplatin, platinum, carboplatin; gemcitabine, paclitaxel, docetaxel, etoposide, vinorelbine, topotecan, or irinotecan; tyrosine kinase inhibitors (e.g., Axitinib, Bosutinib, Cediranib, Dasatinib, Erlotinib, Gefitinib, Imatinib, Lapatinib, Lastaurtinib, Nilotinib, semaxanib, sunitinib, vandetanib, vatalanib, Wortmannin or any other suitable tyrosine kinase inhibitor); apoptosis inducing enzymes, for example TNF polypeptides, TRAIL (TRAIL R1, TRAIL R2), Apoptosis inhibitor 2 (API-2), FasL, Exisulind or other apoptosis inducing enzymes; molecules which hamper cell growth such as 2-Deoxy-D-glucose (2DG), oligomycin, Rapamycin or its analogues, or other chemotherapy agents such as those commonly known to a person skilled in the art. Alternatively they may be anticancer treatments such as radiotherapy, surgical resection, the chemotherapy agents mentioned above or any combination of these.

It is especially advantageous to formulate parenteral compositions in dosage unit form for ease of administration and uniformity of dosage. Dosage unit form as used herein refers to physically discrete units suited as unitary dosages for the mammalian subjects to be treated, each unit containing a predetermined quantity of active material calculated to produce the desired therapeutic effect in association with the required pharmaceutical carrier. The dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

The compositions may be for use in treating cancer. Use includes use of a composition of the invention for the preparation of a medicament or a pharmaceutically acceptable composition for the treatment of cancer. The preparation may further comprise a chemotherapeutic agent for the preparation of a medicament for the treatment of cancer.

Use of the current invention for treatment may result in a significant improvement of glioma patient survival. Preferably, the prognosis for patients and or the survival rate for patients with high-grade gliomas are improved to greater than 50% of those diagnosed with malignant gliomas living 1 year or more after diagnosis, after treatment with the invention. Preferably, patients diagnosed with anaplastic astrocytoma survive more than three years after treatment with the invention. Preferably, Glioblastoma multiforme has a better prognosis with patients living 1 year or more after diagnosis, after treatment with the invention.

Based on our findings, we propose a mechanism by which Ect2 promotes oncogenecity is through regulating the key CDK inhibitor p27^{Kip1}. The finding that Ect2 over-expression de-regulates RhoA activity during cell cycle progression addresses the issue of the absence of activating RhoA mutants in human tumours. Our observations of full-length Ect2 mirror that of Vav1. Thus the manipulation of full length Ect2 protein in this study better reflects the in vivo situation compared to previous models utilizing the truncated forms.

Further, Rac1 and Ect2 co-localize in proliferating glioma cells linking Ect2 targeting with another major oncogene. RAC1 activation (Rac1-GTP pull-down) is induced by exogenous Ect2 expression (data not shown).

In this study, we manipulated Ect2 expression using siRNA and cDNA transfection. p27^{Kip1} levels are inversely replated to Ect2 expression and its associated RhoA activation. Furthermore, we show that the Db1 domain is the minimum motif required for inactivation of the p27^{Kip1} and enhancement of pRb tumour suppressor activity. Our findings indicate that Ect2 may promote cellular transformation and oncogenesis through the inactivation of the p27^{Kip1}-pRb axis. Using human glioma cells, we demonstrate the requirement for Ect2 in G₁/S progression. Ect2 suppression abrogates cell cycle entry of quiescent glioma cells following serum repletion. This is accompanied by high levels of the CDK Inhibitor p27^{Kip1} and reduced Rb hyper-phosphorylation. In contrast, Ect2 over-expression in quiescent cells suppresses p27^{Kip1} and induces serum-independent cell cycle progression. Ect2 modulates p27^{Kip1} through mRNA stability and proteolytic degradation. Furthermore, Ect2 directs Rb hyper-phosphorylation through RhoA. Ect2 over-expression increases RhoA activation, and is parallel with increased binding between Ect2 and activated RhoA. Our findings show that Ect2 oncogenecity is linked to its RhoGEF function in regulating the G₁/S progression through degradation of the key CDK inhibitor p27^{Kip1}. We demonstrate that inactivation of the p27^{Kip1} tumour suppressor is not dependent on N-terminus truncation of Ect2, but is dependent on the DH domain of Ect2. Without being limited to any theory we postulate that Ect2 oncogenecity is linked to its RhoGEF function in regulating the G₁/S progression through degradation of the key CDK inhibitor p27Kip1.

Detailed methods for intended practice with the current invention are described as follows:

Cell Culture and Transfection. A172, U87, U118, U373 and T98G human glioma cells (ATCC, Manassas, Va.) were cultured in Dulbecco's modified Eagle's medium (DMEM) supplemented with 10% fetal bovine serum (Hyclone, South Logan, Utah). Cells were synchronised at quiescence through serum starvation. Full-length human Ect2 was cloned into the expression vector pXJ41 using EcoRI and BamHI digestion. The Ect2 truncation mutants ΔN-Ect2-DH/PH/C, ΔN-Ect2-DH/PH and ΔN-Ect2-DH were cloned in pCTV3-HA3. p27-PF, p27-ApaI, pGVB2 were gifts. For transient expression, cells were seeded at 40% confluence and transfected with 1.5 μg of DNA using Polyfect transfection reagent (Qiagen GmBH, Hilden, Germany). For knockdown experiments, Ect2 siRNAs were transfected into cells using SilentFect (Bio-Rad, Hercules, Calif.). RhoA inhibitor C3 exoenzyme (Upstate Biotech, Billerica, Mass.) was introduced into cells using Lipofectamine2000 (Invitrogen, Carlsbad, Calif.) at a concentration of 10 mg/ml. Cells were treated with the proteasome inhibitor MG132 (Calbiochem, San Diego, Calif.) at a concentration of 100 nM.

Drug treatment: Glioma cells, either mixed culture of synchronized cells will be treated with Temozolomide (TMZ) for 2 h at physiological relevant dose (100 μM) following depletion of MGMT enzyme by O⁶-benzylguanine (Note: MGMT is the major DNA repair enzyme conferring TMZ resistance. MGMT enzyme expression levels vary among different glioma cell lines. Thus, it is essential to deplete it before assessing TMZ cytotoxicity in the context of G₁ growth inhibition). Both TMZ and O⁶-benzylguanine will be dissolved in DMSO, with DMSO final concentration always <0.01% (v/v) in all the treatment.

Proliferation and colony formation assays. Cell proliferation was measured with MTT (3-(4,5-Dimethylthiazole -2-yl)-2,5-diphenyltetrazolium bromide) (Sigma, St. Louis, Mo.) colorimetric assay. Briefly, cells were seeded in 48-well plates and transfected with siRNA the next day. 10 mg/ml MTT reagent was diluted 1:4 with medium and added to cells washed with PBS and allowed to incubate for at least 1 hr at 37° C. Equal-volumes of MTT lysis buffer (50% dimethyl-formamide, 50% water and 20% SDS) were added to solubilise the formazan formed during metabolism. Relative light absorbance at 595 nm was measured using an ELISA plate reader (Dynex Technologies, Chantilly, Va.). To measure long-term proliferation, cells were seeded in 24-well plates and transfected with siRNA the next day. After 24 h, cells were trypsinized and re-plated at a density of 500 cells/well in 6-well plates and assayed for foci formation over 10 days. Colonies were fixed with 4% paraformaldehyde and stained with 1% crystal violet. Representative fields from each well were counted for number of colonies. Student's t-test was used to calculate statistical significance.

Western blot analysis. Whole cell lysates were prepared at the indicated time points following treatment. Cells were lysed in 0.1% SDS buffer (50 mM Tris-HCl, pH7.5, 150 mM NaCl, 1% NP-40, 1% sodium deoxycholate, 0.1 mM sodium vanadate, 50 mM b-glycerophosphate, 1 mM NaF) supplemented with protease inhibitor cocktail (Roche, Basel, Switzerland). Lysates were clarified by centrifugation at 13,000 rpm for 20 min at 4° C. Protein quantity was determined by the Bradford Assay Reagent (Bio-Rad). Typically, 50 μg of protein was resolved on either 6% or 12% SDS-polyacrylamide and transferred onto PVDF membranes (Millipore, Billerica, Mass.). Membranes were probed using antibodies specific to Ect2 and other proteins listed in the attached document. Protein bands were visualized by chemiluminescence using the ECL Western blotting system (Amersham, Arlington Heights, Ill.) according to manufacturer's protocol. Actin was used as loading control.

Rho activity assay. Rho activation was measured using the Rho Activation Assay Kit (Upstate Biotech, Billerica Mass.,) according to manufacturer's protocol. Briefly, cells were lysed in 1×Mg2+-containing lysis buffer and clarified by centrifugation at 13,000 rpm for 20 min at 4° C. 3-500 mg of lysate was incubated with 20 mg of the Rhotekin RBD agarose beads and rotated for 1 hr at 4° C. Beads were washed three times with lysis buffer and released by boiling in 1×SDS sample buffer supplemented with 10 nM DTT. RhoA activation and total RhoA amount were determined by Western blotting with RhoA antibody (Santa Cruz).

Cell Cycle Analysis and BrdU incorporation. Cell pellets were collected at the time points indicated after treatment and fixed in 70% ethanol at −20° C. Before analysis, cells were washed and rehydrated with phosphate buffered saline and stained with 50 mg/ml propidium iodide supplemented with 100 mg/ml RNase A. For BrdU incorporation, cells were pulsed with 10 mM BrdU and fixed in 70% ethanol before staining. 2N HCl was used for denaturation before incubation with anti-BrdU antibody conjugated with FITC. Subsequent analysis was carried out on the FACS Calibur using the CellQuest Pro software (Becton Dickinson, Franklin Lakes, N.J.).

cDNA Synthesis and Real-Time RT-PCR. RNA was extracted from cell pellets using Tri Reagent (Molecular Research Centre, Cincinnati, Ohio) according to manufacturer's protocol. 500 ng of total RNA was used for 1st strand cDNA synthesis using Improm-II reverse transcriptase (Promega, Madison, Wis.) and the 1st strand cDNA was subsequently used as the template for Real-Time RT-PCR. The following primers were used for detecting p27Kip1 mRNA levels, sense: 5′-AAC CGA CGA TTC TTC TAC TC-3′ and anti-sense: 5′-GAT GTC CAT TCC ATG AAG TC-3′. Briefly, 2 μl of cDNA template and 1 μM of each primer were added to Quantitect SYBR Green PCR mix (Qiagen) and allowed to cycle on the DNA Engine Opticon (Bio-Rad) at the following parameters: a pre-denaturing step at 95° C. for 10 min, followed by 45 cycles of denaturation at 95° C. for 1 min, annealing at 55° C. for 30 sec, and extension at 72° C. for 1 min. Duplicate experiments were carried out. Absolute amounts of transcripts were calculated using CT values obtained and normalized against actin control. Student's t-test was used to calculate statistical significance.

Assay of mRNA stability. Cells were treated with Actinomycin D (2.5 mg/ml) and total RNA was harvested at the time points indicated. Abundance of p27^{Kip1} transcripts were determined by Realtime PCR and normalized against that of actin control.

Luciferase reporter assay. Cells were co-transfected with the indicated reporter plasmids and pTKRL expressing Renilla luciferase at a ratio of 1:20. Measurement of promoter activity was carried out using the Dual Luciferase Assay kit (Promega) according to manufacturer's protocol.

Autophagy analysis: A) Detection of AVO (autophagy) by live cell staining with acridine orange: Cells will be incubated with acridine orange (at a final concentration of 1 μg/ml) for 15 min in CO₂ incubator at 37° C. Cells will then be collected by trypsinization and resuspended in phenol-red free growth medium and analysed by FACS for FL1 and FL3. B) LC3-II detection: Microtubule-associated protein 1 light chain 3 (LC3), a homologue of Apg8p essential for autophagy in yeast, is associated to the autophagosome membranes after processing. Two forms of LC3, LC3-I and LC3-II, are produced post-translationally in various cells. LC3-I is cytosolic, whereas LC3-II is membrane bound. The presence of LC3-II and its punctate cytosolic distribution have been widely used as an autophagy marker. In our study, the presence of LC3-II (15˜16 kDa) will be detected by Western blotting. The punctate cytosolic distribution of LC3-II will be analysed with our established stable glioma cell lines (A172, U87, U118, U373 and T98G) expressing EGFP-LC3 fusion protein.

### Example 1

### Down-Regulation of Ect2 Impedes G₁/S Cell Cycle Progression.

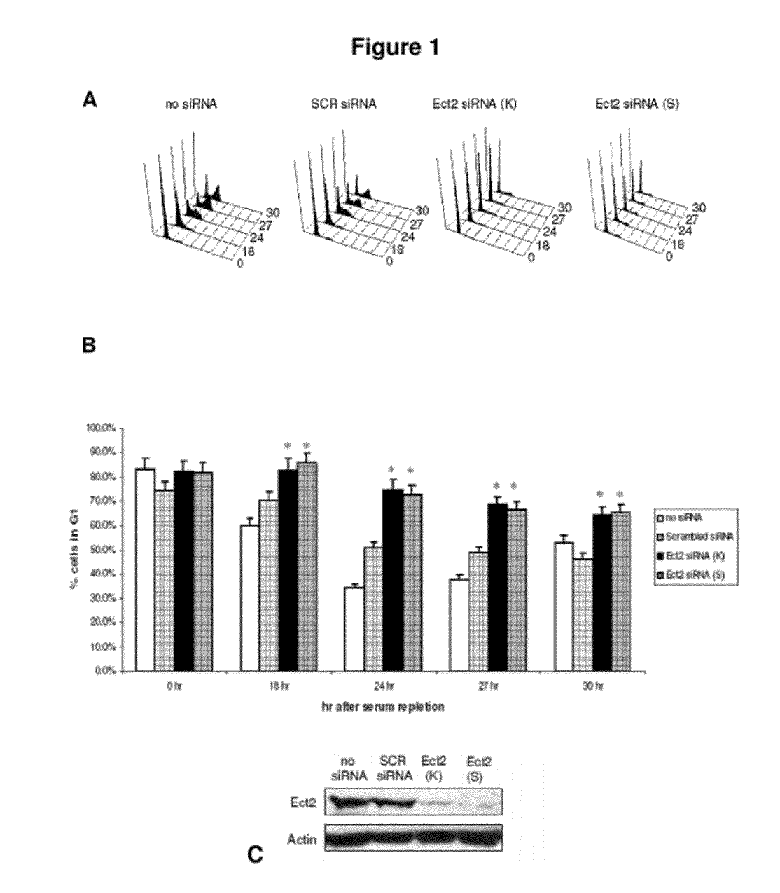

To clarify the role of Ect2 In cell cycle regulation, quiescent human glioma cells were transfected with Ect2 siRNA and analysed for cell cycle progression following serum repletion. In non-transfected and scrambled sequence siRNA transfected cells, DNA synthesis was initiated at around 18 h with S phase peaked at around 24 h. The G₂/M boundary was crossed between 27 to 30 h (FIG. 1a). In contrast, cells transfected with either of the two Independent Ect2 siRNAs showed a prominent accumulation of cells in G₁ phase, persisting up to 30 h after serum repletion. In particular, Ect2 siRNA transfected cells contained nearly 73% of cells at G₁ phase, compared to 35% in non-transfected cells and 50% in scrambled sequence control transfected cells 24 h after serum repletion (p<0.05) (FIG. 1b). This demonstrates that Ect2 is required for G₁/S progression.

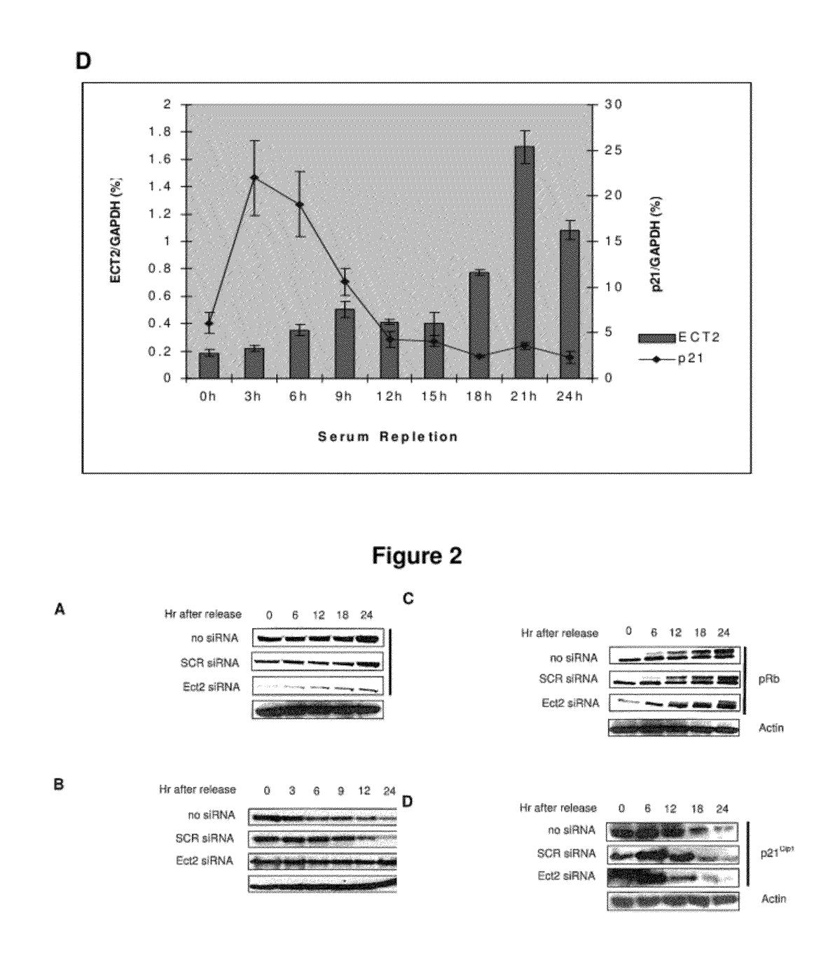

Ect2 down regulation-induced G₁ arrest is accompanied by increase in p27^{Kip1} and decrease in Rb phosphorylation. Expression of the CDK inhibitor p27^{Kip1} is elevated during quiescence and its degradation is required for cell cycle re-entry and subsequent G₁/S progression. We investigated whether down-regulation of Ect2 altered p27^{Kip1} protein levels. Expression of Ect2 protein was inhibited following siRNA transfection compared to non-transfected and scrambled sequence-transfected cells (FIG. 2a). In non-transfected and scrambled sequence-transfected cells, the levels of p27^{Kip1} gradually decreased as quiescence-synchronised cells re-entered the cell cycle and progressed into S-phase upon serum repletion. In contrast, p27^{Kip1} protein levels in Ect2 siRNA-transfected cells remained high and persisted till 24 h following serum repletion (FIG. 2b). In contrast, the other CDK inhibitor p21^{cip1} did not change with the manipulation of Ect2 protein expression (FIG. 2d).

Phosphorylation and inactivation of Rb protein is critical for G₁/S progression. Thus, we determined the phosphorylation status of Rb in Ect2 down-regulated human glioma cells. Rb phosphorylation was defined by mobility shift. During quiescence, Rb was present as the hypo-phosphorylated form in control and scrambled sequence-transfected cells. The hyper-phosphorylated form appeared at 6 h following serum repletion and peaked at 24 h when cells were entering S phase. In contrast, Rb phosphorylation was significantly delayed in Ect2 siRNA-transfected cells, with no detectable Rb hyper-phosphorylation until 12h following serum repletion (FIG. 2c). After 12 h, Rb phosphorylation in Ect2 siRNA-transfected cells was also significantly lower than the level observed in control cells. These demonstrate that Rb hyper-phosphorylation is greatly impaired throughout G₁ phase by Ect2 down-regulation. p21^{Cip1} protein abundance did not fluctuate with Ect2 suppression (FIG. 2d).

Ect2-mediated p27^{Kip1} suppression is serum-independent. Our data show that Ect2 down-regulation in serum-free conditions impaired cell cycle re-entry and inhibited G₁ progression through increased p27^{Kip1} protein levels.

### Example 2

### Ect2 Over-Expression Induces Cell Cycle Progression.

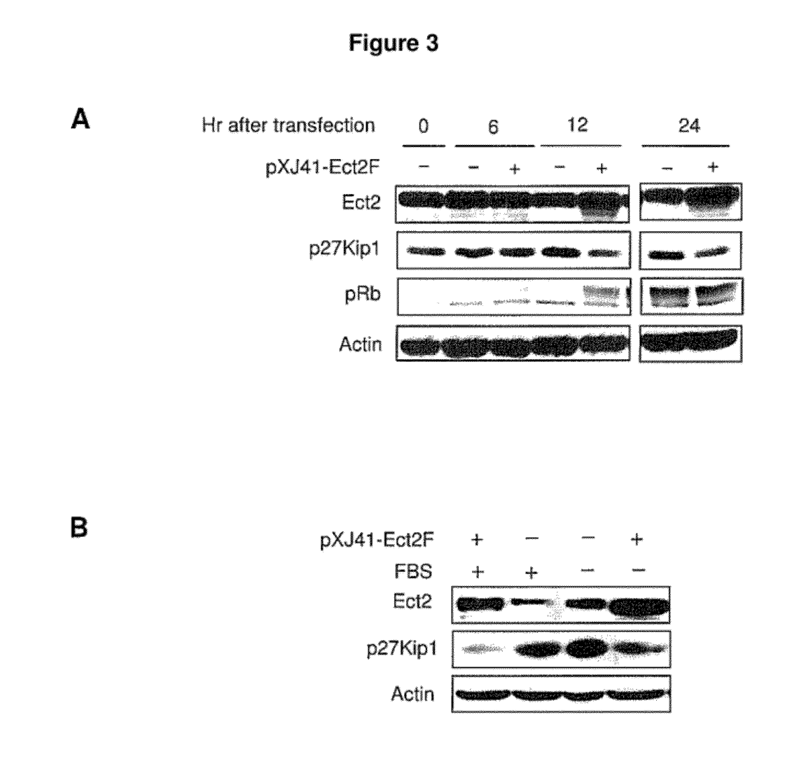

We examined if Ect2 over-expression suppressed p27^{Kip1}. Full-length Ect2 was over-expressed in asynchronous glioma cells. At 6 h of cDNA transfection, the level of p27^{Kip1} protein showed a decreasing trend whereas the level of p27^{Kip1} remained unchanged in non-transfected samples. We also observed correspondingly increased Rb phosphorylation following Ect2 over-expression (FIG. 3a). A plateau of Rb phosphorylation was reached at 24 h for both Ect2 transfected and control cells. Next, we asked if the suppression of p27^{Kip1} by Ect2 was serum-dependent. Cells were starved and transfected with the Ect2. p27^{Kip1} was suppressed in Ect2 transfected cells regardless of the presence of serum (FIG. 3b). These results show that Ect2 suppresses p27^{Kip1} protein and promotes Rb phosphorylation independent of serum.

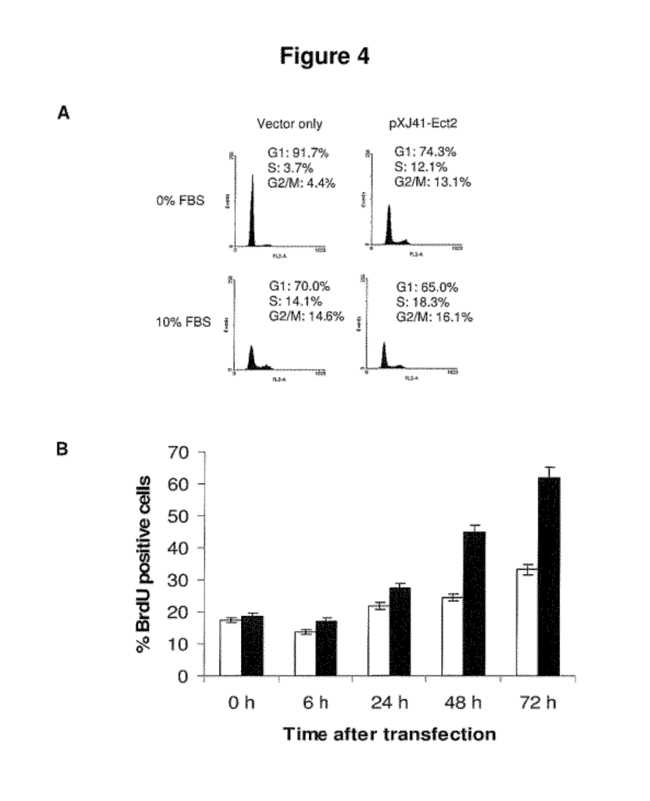

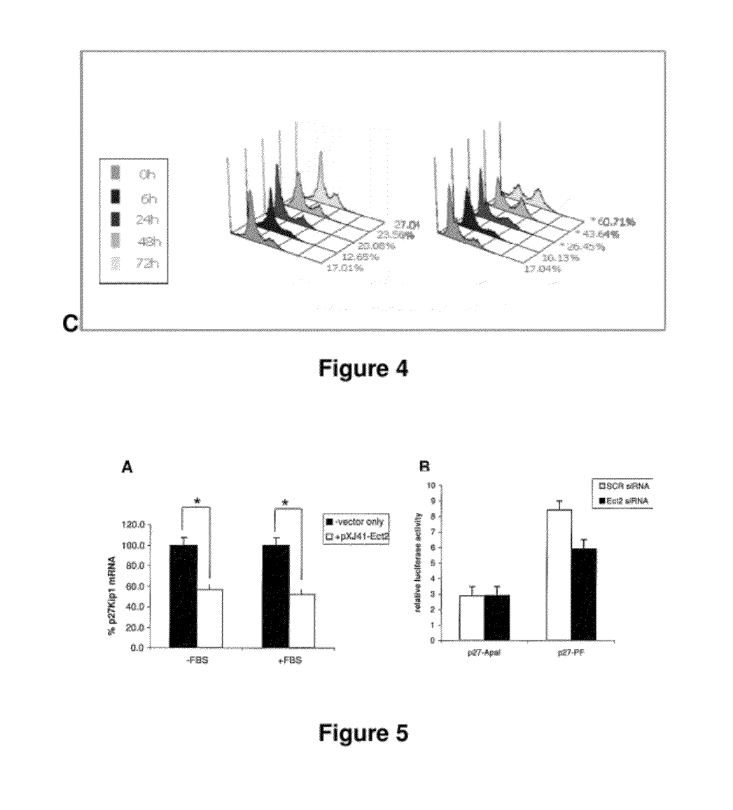

Suppression of p27^{Kip1} and hyper-phosphorylation of Rb had profound effects on cell cycle progression. To determine the effects on cell cycle progression, Ect2 expression was induced in quiescent glioma cells. Under serum-free conditions, cells expressing exogenous Ect2 contained a higher percentage of S-phase cells than the vector-transfected control (12% vs. 3%) (FIG. 4a). This Ect2-induced increase in S-phase population was also observed in non-starved cells, although the effect was partially masked by the presence of relatively high basal level of S-phase cells in the control (FIG. 4a).

To analyse the effect of Ect2 over-expression on DNA synthesis, quiescent glioma cells were incubated with BrdU, and BrdU incorporation was followed up 0 to 72 h following Ect2 transfection. Cells over-expressing Ect2 contained a markedly higher percentage of BrdU-positive cells than control cells (61% vs. 27%), demonstrating that Ect2 over-expression induces serum-independent DNA synthesis in quiescent glioma cells (FIG. 4B).

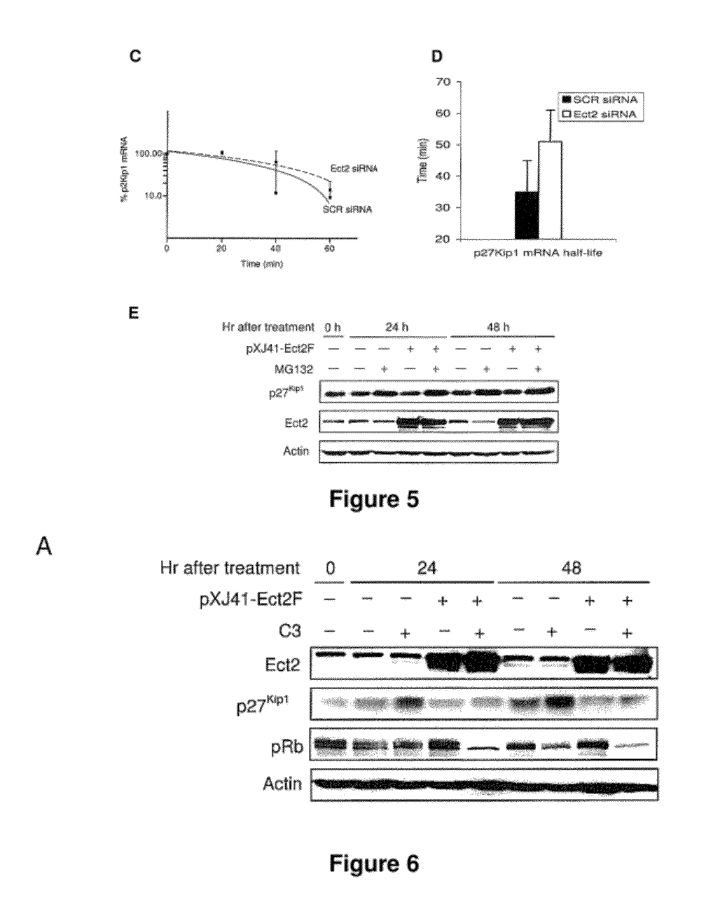

Ect2 regulates p27^{Kip1} abundance at mRNA and protein levels. To determine how p27^{Kip1} is regulated by Ect2, we measured the amount of p27^{Kip1} transcripts by Real-Time RT-PCR. p27^{Kip1} mRNA levels decreased upon over-expression of Ect2 regardless of the presence of serum (FIG. 5a). To verify transcriptional regulation, luciferase reporters (fused to full-length or truncated p27 promoters) were used. Ect2 siRNA transfection failed to increase p27^{Kip1} promoter activity and further inhibited p27^{Kip1} promoter activity (FIG. 5b). Thus, it is not likely that Ect2 down-regulates p27^{Kip1} mRNA at the level of transcriptional regulation.

Since p27^{Kip1} abundance could also be influenced by the stability of mRNA, we asked if Ect2 down-regulation prolonged the half-life of p27^{Kip1} mRNA. Cells transfected with control or Ect2 siRNA were treated with Actinomycin D to inhibit de novo transcription. p27^{Kip1} mRNA was quantified at various time points with Real-Time RT-PCR. The half-life of p27^{Kip1} mRNA increased from 35 min in scrambled sequence control siRNA-transfected cells to 51 min in Ect2 siRNA-transfected cells (p<0.05) (FIG. 5c-d). Our results demonstrate that Ect2 down-regulation prolongs the half-life of p27^{Kip1} mRNA.

We subsequently tested the requirement of proteasome in Ect2-mediated p27^{Kip1} suppression by using the proteasome inhibitor MG132. Over-expression of Ect2 alone was sufficient to reduce p27^{Kip1} protein to below basal levels in control cells. However, addition of MG132 to cells over-expressing Ect2 completely abrogated the suppression of p27^{Kip1} (FIG. 5e), indicating that Ect2 is dependent on the proteasome for p27^{Kip1} regulation.

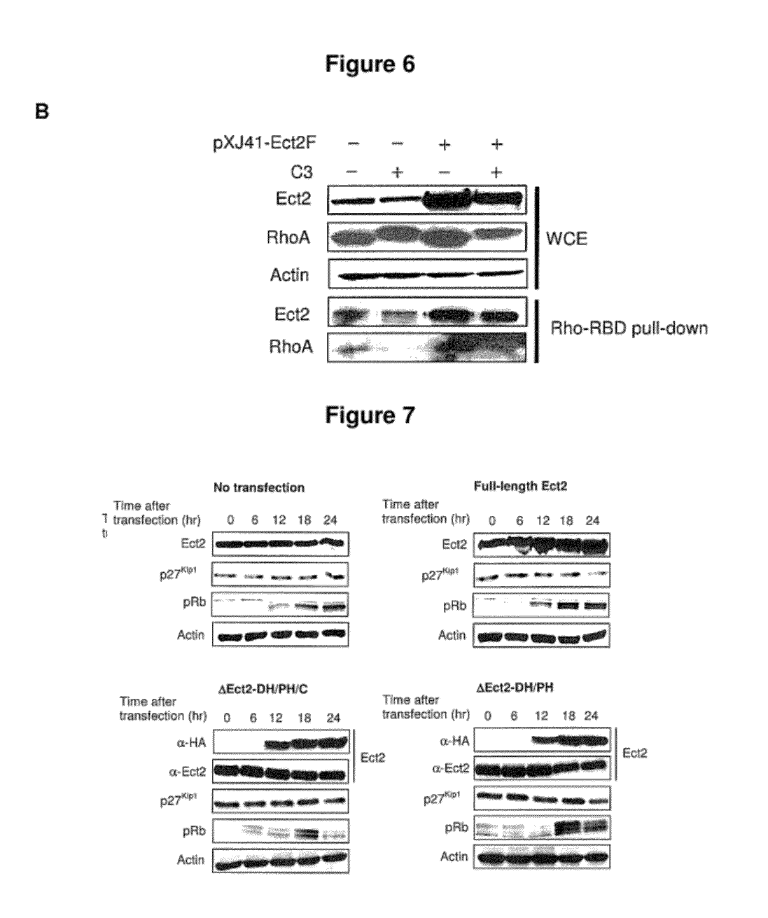

Ect2 promotes G₁/S progression through the small GTPase RhoA. RhoA mediates G₁/S progression through suppression of p27^{Kip1}. However, its activating GEF is unidentified. Since Ect2, as a RhoGEF, suppresses p27^{Kip1} to promote G₁/S progression, we examined if RhoA activity is required for these events. Over-expression of Ect2 suppressed p27^{Kip1} and increased Rb hyper-phosphorylation. Incubation with RhoA specific inhibitor C3 in cells over-expressing Ect2 partially restored p27^{Kip1} protein level and completely suppressed Rb hyper-phosphorylation (FIG. 6a).

We further defined the relationship between RhoA activation and Ect2 over-expression by performing a Rho activation assay. In the presence of C3, RhoA activation was abrogated (FIG. 6b). RhoA activity Increased significantly following Ect2 over-expression and C3 failed to attenuate RhoA activity at the concentration tested. Furthermore, Ect2 association with activated RhoA increased with Ect2 over-expression. The presence of C3 slightly reduced the amount of Ect2 associated with activated RhoA. Our results show that Ect2 over-expression increases RhoA activation and this relationship is highlighted by the interaction between Ect2 and activated RhoA.

Over-expression of the full-length Ect2 protein resulted in hyper-induction of the primary vulva fate specification at G₁, a process that is dependent on Ras and Rho-1 activity Thus, the activation of proliferative signalling pathways by Ect2 the result of an increase in intrinsic Ect2 activity. Our data demonstrating increase in activated RhoA and inhibited p27^{Kip1} tumour suppressor pathway following full-length Ect2 over-expression supports this hypothesis, and provides a possible mechanism for the role of full-length Ect2 in regulating the G₁/S progression as well as in malignant transformation.

### Example 3

The DH Domain is Required for Suppression of p27^{Kip1} by Ect2.

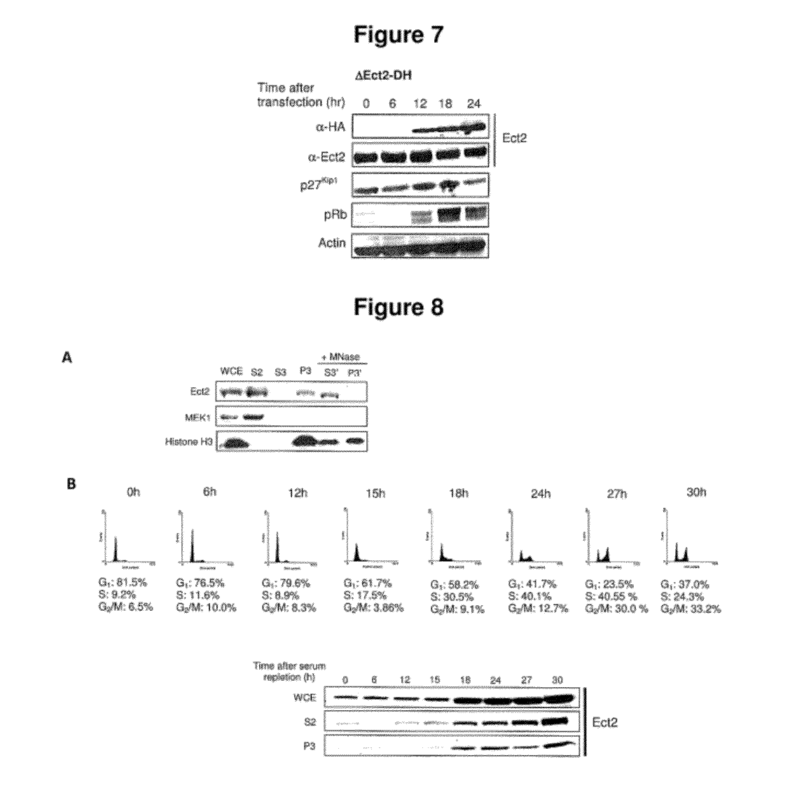

The N-terminal truncated form of Ect2 induces malignant transformation in mouse fibroblasts with an unknown mechanism of oncogenicity. We investigated whether the deletion of N-terminal regions affected Ect2-mediated Rb phosphorylation and suppression of p27^{Kip1}. p27^{Kip1} protein levels decreased, whereas Rb phosphorylation enhanced in cells over-expressing both full-length Ect2 and its various truncation mutants (FIG. 7). Particularly, cells expressing only the DH domain (ΔEct2-DH) exhibited the same pattern of p27^{Kip1} suppression and pRb hyper-phosphorylation as the full-length, ΔN-Ect2-DH/PH/C or ΔN-Ect2-DH/PH albeit at a slightly lower level of expression. These results show that the DH domain is the minimal functional motif required for Ect2 to suppress p27^{Kip1}. Neither PH domain nor N-terminal (BRCT domain) truncation is necessary for such regulation.

Ect2 is found in the cytoplasm during quiescence.

Previously Ect2 was reported to be present in the nucleus during interphase and dispersed to the cytoplasm during mitosis. This creates a conundrum whereby the cellular location of Ect2 contradicts its activation of RhoA during G₁/S demonstrated earlier. To address this, we analysed the location of Ect2 in U118 glioma cells. Ect2 was found in both cytoplasmic and nuclear fractions during interphase (FIG. 8a). Further tracking of Ect2 localization as quiescent cells were stimulated to re-enter cell cycle revealed that low amounts of the protein was present in the cytoplasm during G₀/G₁-phase (FIG. 8b). The cytoplasmic fraction increased as cells progressed towards mitosis. This finding, although contradictory to previous studies showing the unique localization of Ect2 in the nucleus during G₁, may resolve the Issue of how Ect2 is able to activate cytoplasmic RhoA during quiescence.

### Example 4

### Ect2 Nucleolus Distribution, DNA Binding and Regulation of Ribosome Biogenesis

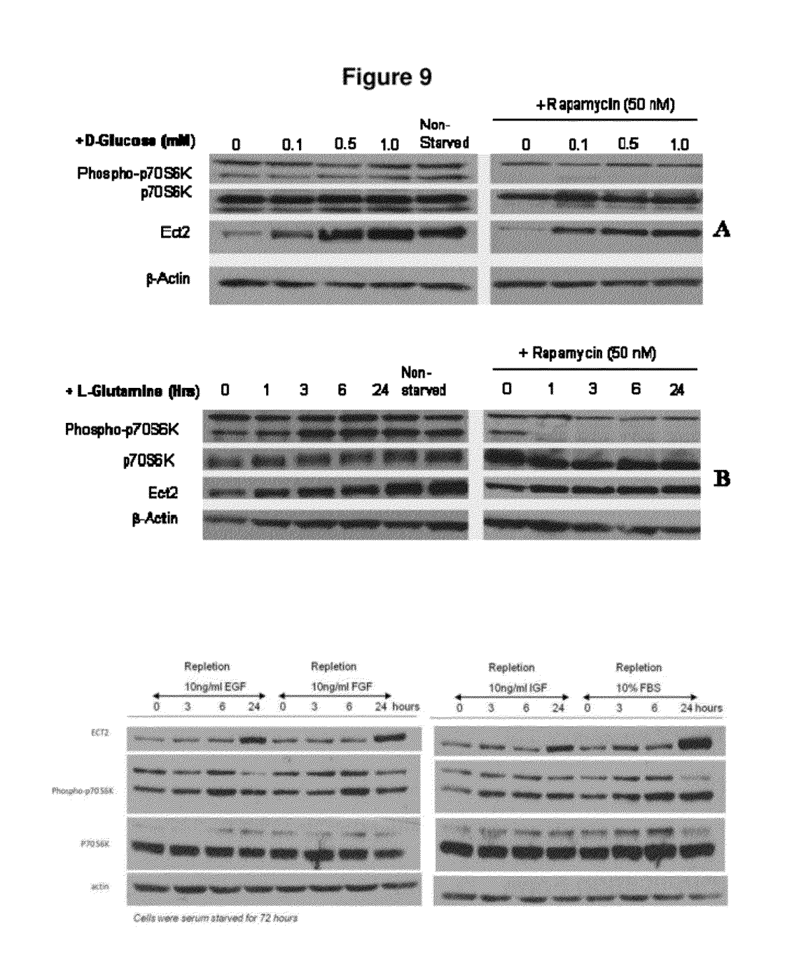

Further to the identification of the role in G₁/S cell cycle regulation, we found that Ect2 itself was regulated by mTOR as well as typical mTOR upstream inputs (e.g. nutrients) (FIG. 9). U118 glioma cells were serum-starved for 72 hr then repleted with 10 ng/ml of Epithelial/Fibroblast/Insulin-like growth factors (EGF/FGF/IGF) or 10% Fetal bovine serum (FBS). ECT2 expression increased significantly at 24 hr with a corresponding increase in mTOR activity as measured by p70S6 kinase phosphorylation (FIG. 9C).

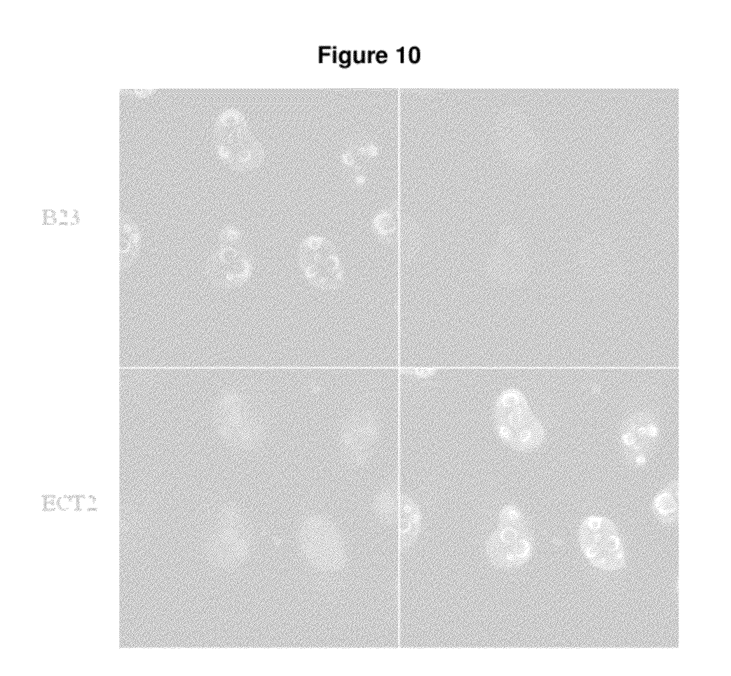

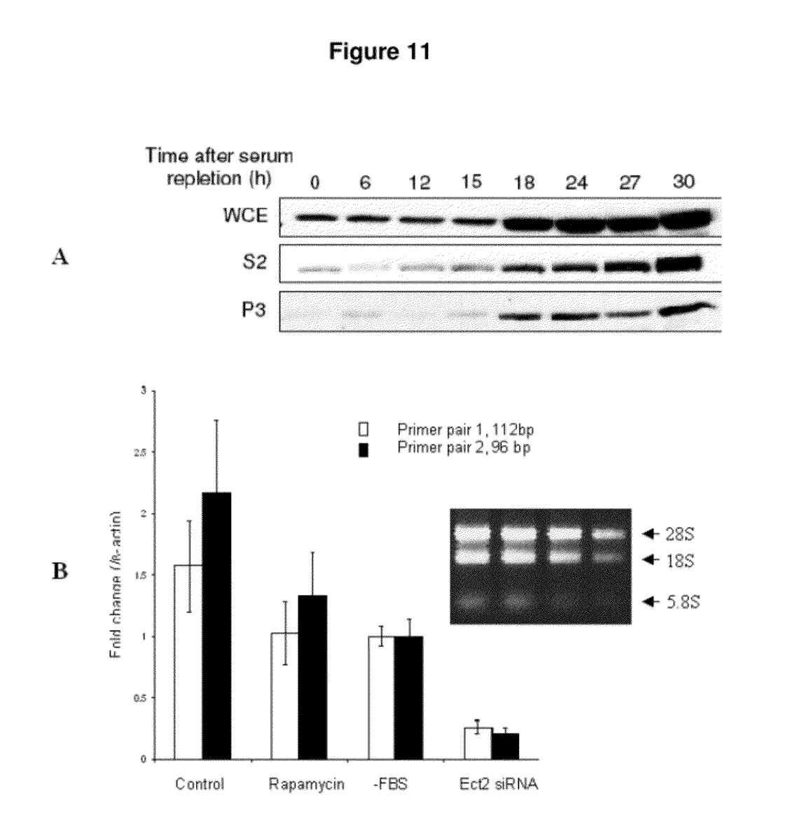

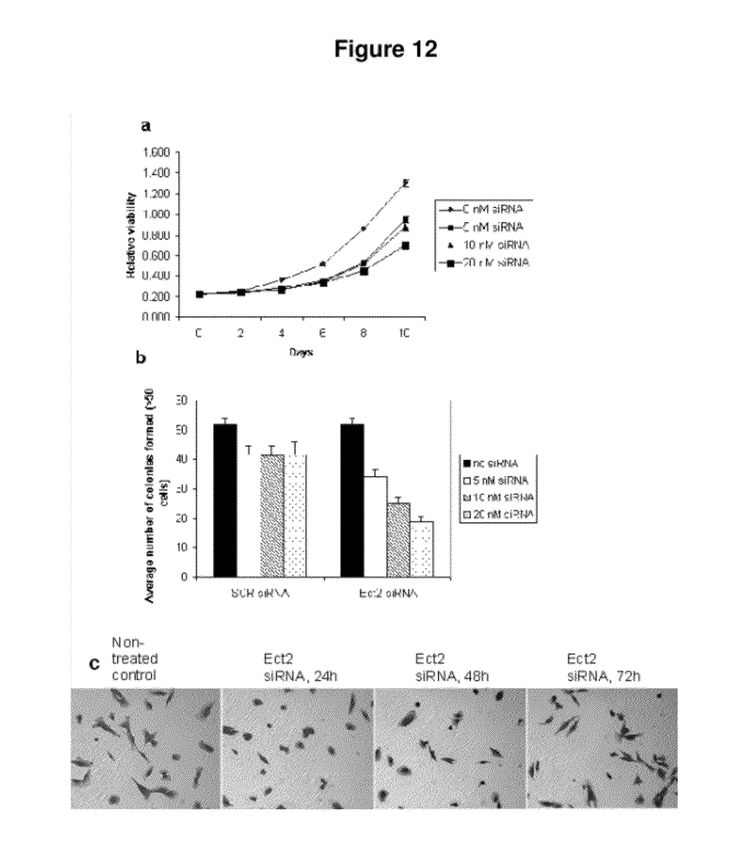

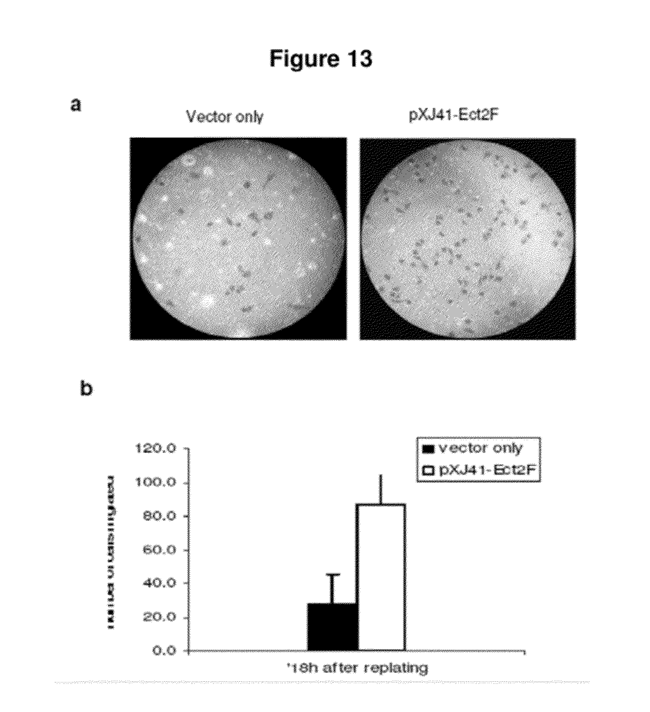

Such finding indicates that Ect2 may be involved in cell growth pathways. In particular, it may promote G₁-phase cell growth and eventually leads to G₁/S transition. In accordance with this hypothesis, we found that Ect2 had a nucleolar distribution (Note that the nucleolus is the ‘factory’ of ribosome biogenesis, where pre-rRNA is transcribed from rDNA and processed into ribosome particles) Serum starvation for 48 hours induced nucleolar accumulation of ECT2 (FIG. 10). Furthermore, Ect2 was associated with chromatin fraction and knockdown of Ect2 dramatically down regulated pre-rRNA transcription, a rate-limiting step in ribosome biogenesis (FIG. 11). Cell cycle-dependent ECT2-DNA binding was observed in WCE, whole cell lysate; S2, cytoplasm; P3, chromatin fractions (FIG. 11 A). ECT2 was shown to affect ribosome biogenesis through regulating pre-rRNA transcription as verified by quantitative real-time RT-PCR with two independent primers (FIG. 11B). Ribosome biogenesis, which is often up-regulated in tumour cells, is essential for cell growth. We found that Ect2 knockdown not only affected ribosome biogenesis, but also significantly inhibited cells growth, including growth rate (FIG. 12a), colony formation (FIG. 12b) and cell size (FIG. 12c)

### Example 5

Methods of Inhibiting Glioma Growth with Combined Application of Ect2 Inhibitors and Chemotherapeutic Agents.

Treatment of glioma growth was significantly improved through combined Ect2 and chemotherapeutic agents that act on glycolysis (Warburg effect)/mTOR inhibition. Growth inhibition was induced by Ect2 knockdown or glycolysis and RTK/PI3K/Akt/mTOR inhibitors (FIG. 20A). Ect2 knockdown significantly enhances glycolysis and RTK/PI3K/Akt/mTOR inhibitor-induced growth inhibition (FIG. 20B). Ect2 knockdown further enhances combined inhibition induced by glycolysis and RTK/PI3K/Akt/mTOR inhibitors (FIG. 20C).

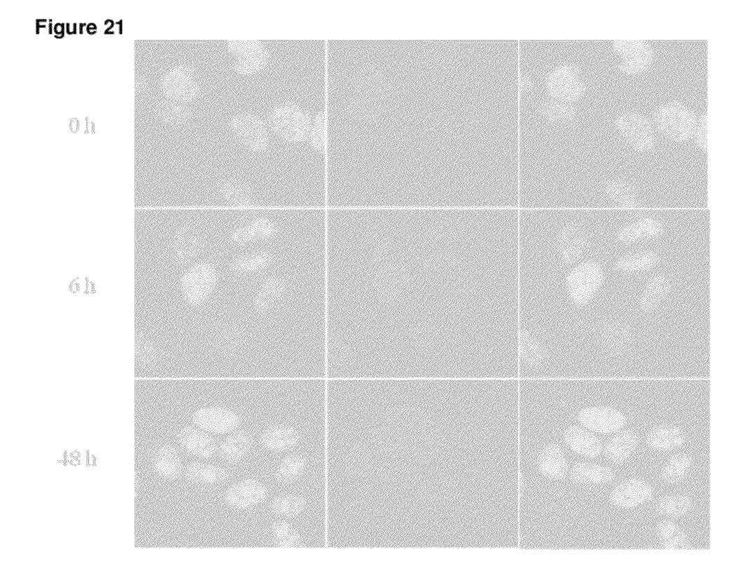

Glucose-induced ribosome biogenesis (as verified by real-time RT-PCR quantification of 45S pre-rRNA) is dependent on ECT2. U118 glioma cells were treated with glycolysis inhibitor, 2-DG (10 mM) in the presence of glucose (5 mM). Ribosome biogenesis inhibition was reversed by supplying high concentration of glucose (25 mM), and was independent on ECT2. Real time-PCR quantification of 45S pre-rRNA to assess the effect of ECT2 knockdown on ribosome biogenesis. U118 glioma cells showed an increase in pre-rRNA level (important pre-cursor for ribosome biogenesis) after glucose repletion whereas this increase is suppressed in cells with ECT2 knockdown (FIG. 20D). Rapamycin (50 nM) induces time-dependent nucleolar accumulation of Ect2. Rapamycin induces nucleolar sequestration of Ect2 (FIG. 21). In each experiment the following drug concentrations were used in each experiment; 10 μM of Erlotinib, 5 μM Wortmannin, 10 μM API-2, 50 nM Rapamycin, 10mM2DG and 2 μg/ml of oligomycin.

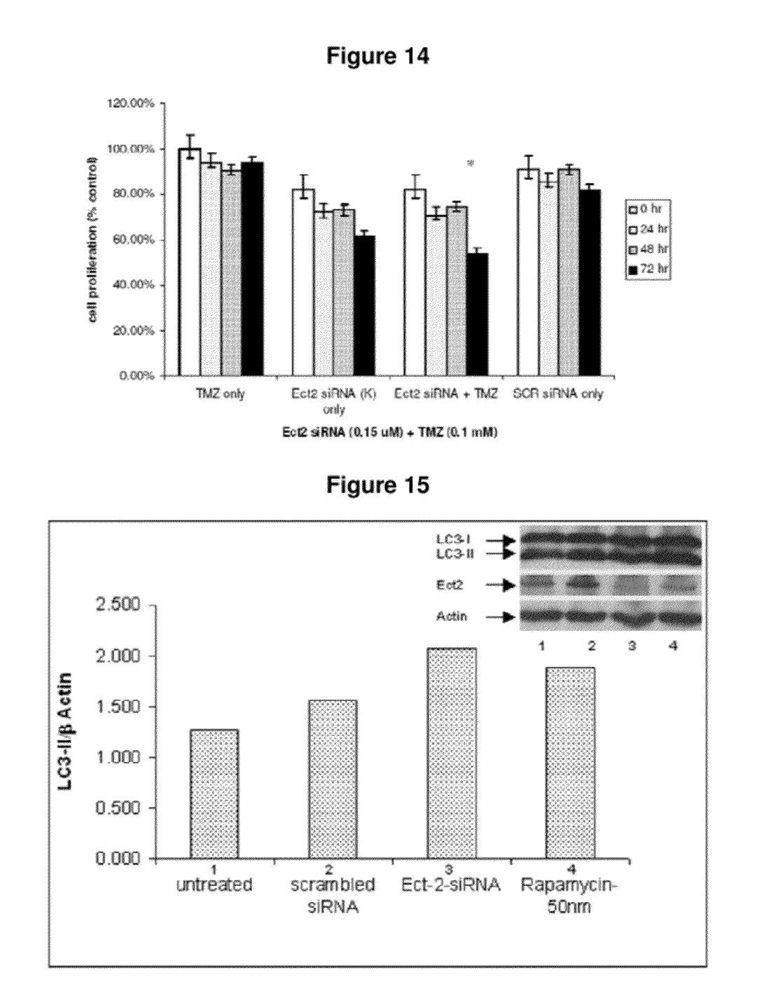

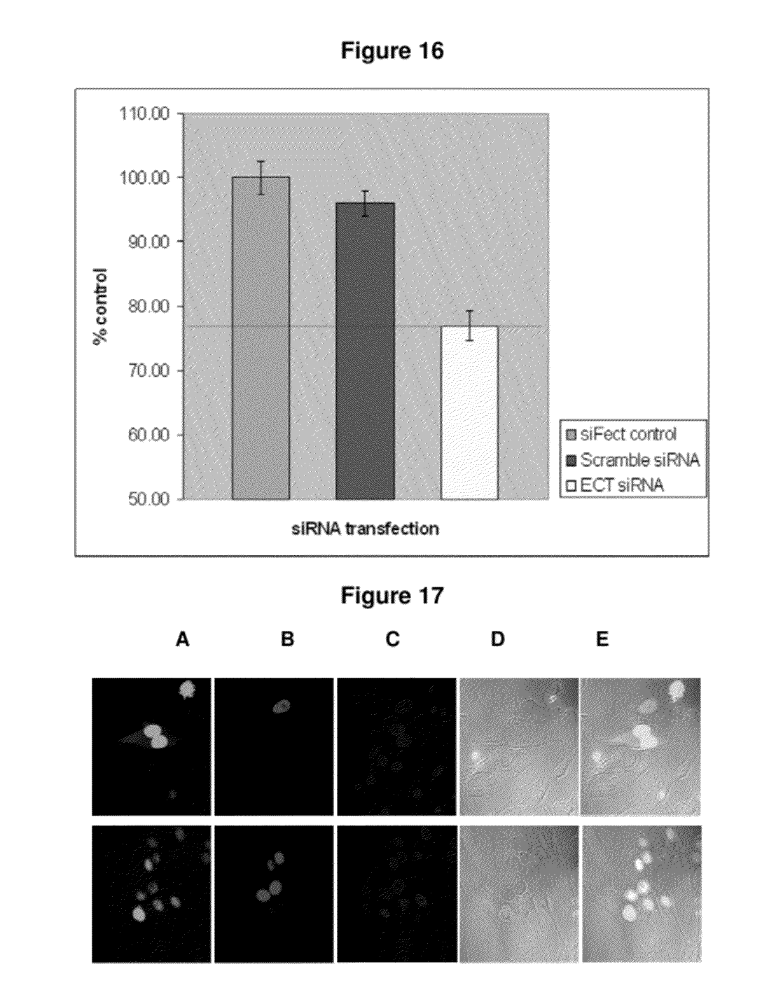

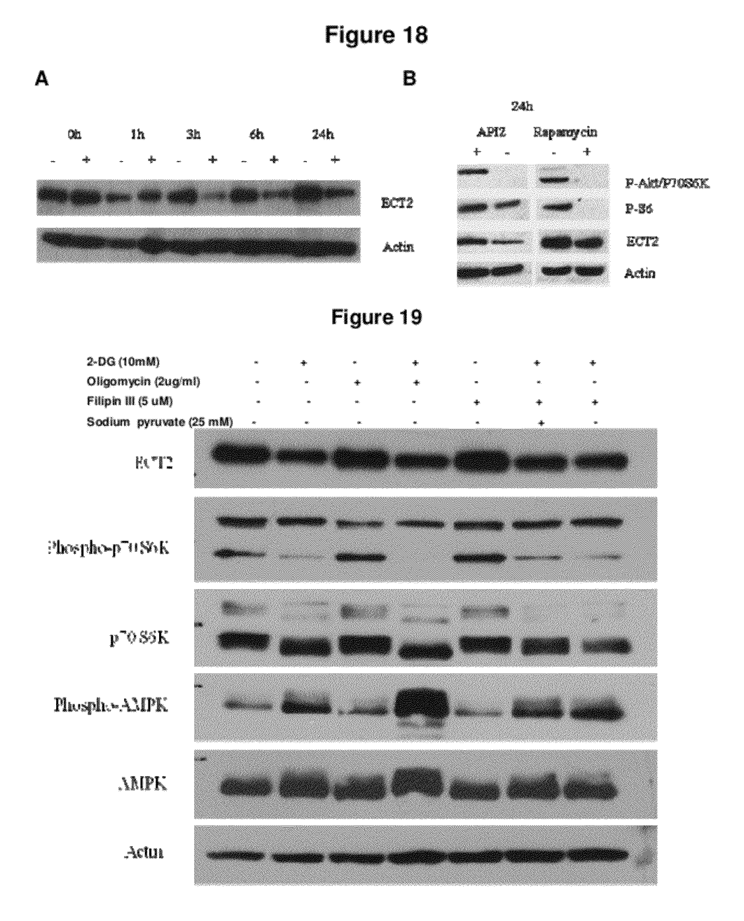

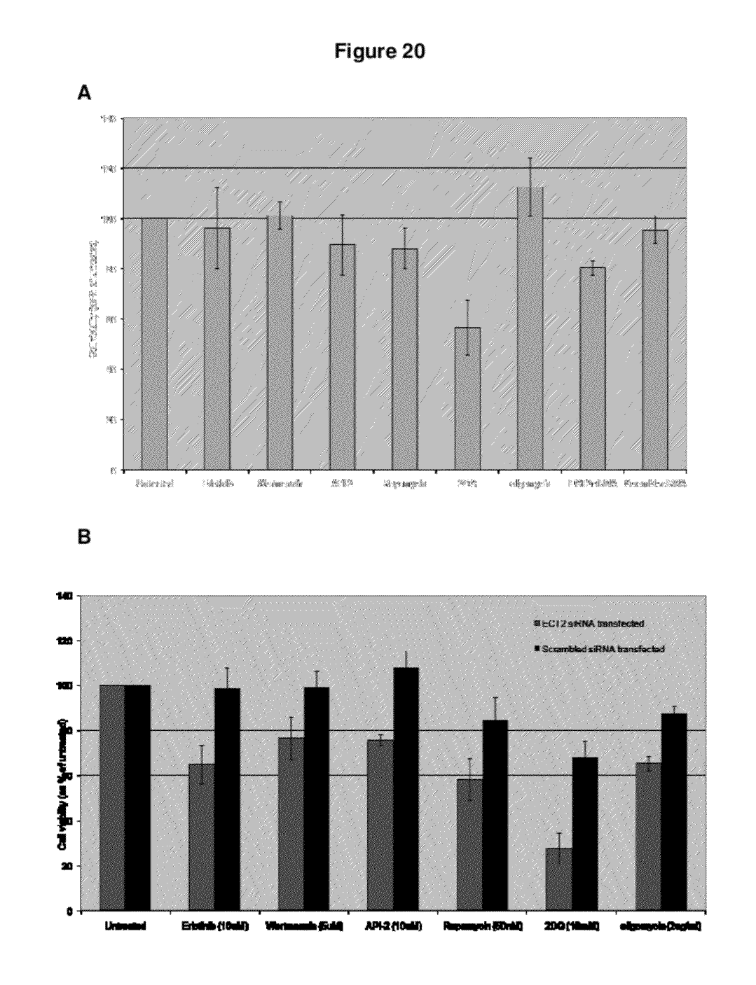

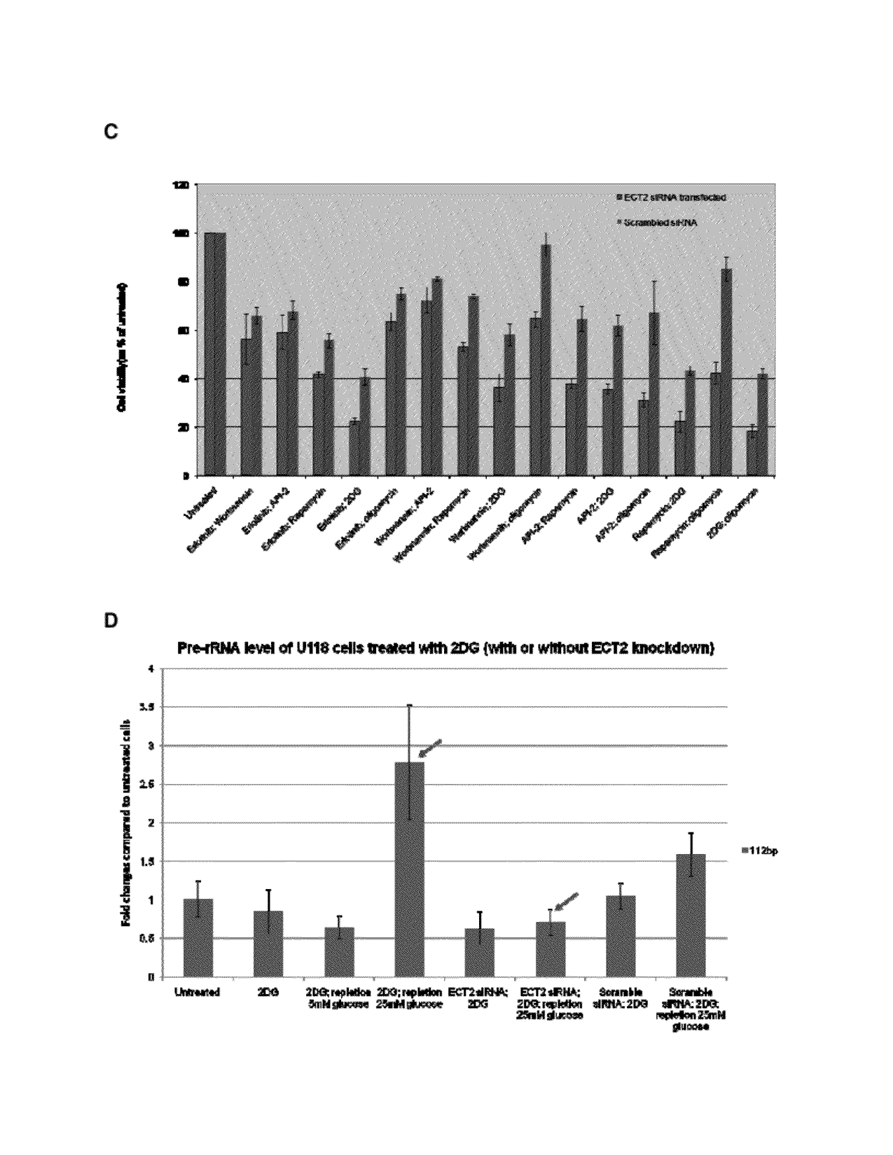

The use of ECT2 inhibitor sensitizes glioma cell stochemotherapy treatment such as with Temozolomide (FIG. 14). ECT2 regulation by RTK/mTOR pathway through EGFR inhibition (FIG. 18) with Erlotinib (10 uM) (FIG. 18A), Akt inhibition with API2 (10 uM), and mTOR inhibition with Rapamycin (50 nM) (FIG. 18B) Warburg effect and ECT2 regulation was examined in U118 glioma cells. The cells were treated with glycolysis or oxidative phosphorylation inhibitors [2-Deoxy-glucose (2DG) and oligomycin, respectively]. Filipin III was used to permeabilise mitochondria to provide exogenous source of pyruvate for the Tricarboxylic acid (TCA) cycle. Cellular energy level was measured by Western blot analysis of AMPK phosphorylation. Cells with glycolysis inhibition showed higher level of phosphorylated AMPK, significantly lower mTOR activity and reduced ECT2 expression whereas oxidative phosphorylation inhibtion did not affect mTOR activity or ECT2 expression (FIG. 19).

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

What is claimed is:

1-35. (canceled)

36. A method of inhibiting cell growth and cell cycle progression from G1 to S phase and increasing protein expression of p27^{kip1} by removing, degrading or neutralising the concentration of epithelial cell transforming sequence 2 (Ect2) in a cellular environment.

37. The method as claimed in claim 36 wherein the cellular environment is in vitro.

38. The method as claimed in claim 36 wherein the cellular environment is in vivo.

39. The method as claimed in claim 38 wherein the cellular environment is in a glioma tissue.

40. The method of claim 36 wherein the concentration of Ect2 is removed, degraded or neutralised by an siRNA.

41. The method of claim 40 wherein the siRNA comprises SEQ ID NO: 1 or SEQ ID NO: 2 or SEQ ID NO: 3.

42. The method of claim 36 wherein the concentration of Ect2 is removed, degraded or neutralised by an Ect2 specific antibody which antibody comprises a sequence capable of binding selectively to a sequence set out in SEQ ID NO: 4 or SEQ ID NO: 5.

43. The method of claim 42 wherein the antibody is catalytic.

44. The method of claim 36 further comprising adding a chemotherapeutic agent to the cellular environment.

45. A method for treating a patient to at least reduce glioma growth, which comprises the step of:
a. contacting the glioma with an antagonist to epithelial cell transforming sequence 2 (Ect2) wherein cell growth and cell cycle progression from G1 to S phase in the cells of the glioma are inhibited and protein expression of p27^{kip1} is increased

46. The method of claim 45 wherein the antagonist is an siRNA.

47. The method of claim 46 wherein the siRNA comprises SEQ ID NO: 1 or SEQ ID NO: 2 or SEQ ID NO: 3.

48. The method of claim 45 wherein the antagonist is an Ect2 specific antibody which antibody comprises a sequence capable of binding selectively to a sequence set out in SEQ ID NO: 4 or SEQ ID NO: 5.

49. The method of claim 45 wherein the antagonist engages the DH domain of Ect2 which antibody comprises a sequence capable of binding selectively to a sequence set out in SEQ ID NO: 5.

50. The method of claim 45 further comprising adding a chemotherapeutic agent to the glioma.

51. A composition comprising an antagonist of cell growth and cell cycle progression from G1 to S phase and an agonist of p27^{kip1} protein expression capable of removing, degrading or neutralising the concentration of epithelial cell transforming sequence 2 (Ect2).

52. The composition of claim 51 wherein the antagonist comprises a therapeutically effective amount of the antagonist to Ect2.

53. The composition of claim 51 wherein the antagonist is an siRNA.

54. The composition of claim 53 wherein the siRNA comprises SEQ ID NO: 1 or SEQ ID NO: 2 or SEQ ID NO: 3.

55. The composition of claim 51 wherein the antagonist is an antibody to Ect2 which antibody comprises a sequence capable of binding selectively to a sequence set out in SEQ ID NO: 4 or SEQ ID NO: 5.

56. The composition of claim 55 wherein the antibody is a catalytic antibody to Ect2.

57. The composition of claim 51 wherein the antagonist engages the DH domain of Ect2.

58. The composition of claim 51 for use as a medicament for treating a patient with cancer.

59. The composition of claim 51 for use as a medicament for treating a patient with glioma.

60. The composition of claim 51 further comprising a chemotherapeutic agent.

61. The composition of claim 60 wherein the chemotherapeutic agent is selected from: Temozolomide; cisplatin, platinum, carboplatin; gemcitabine, paclitaxel, docetaxel, etoposide, vinorelbine, topotecan, or irinotecan; tyrosine kinase inhibitors Axitinib, Bosutinib, Cediranib, Dasatinib, Erlotinib, Gefitinib, Imatinib, Lapatinib, Lastaurtinib, Nilotinib, semaxanib, sunitinib, vandetanib, vatalanib, Wortmannin; apoptosis inducing enzymes, TNF polypeptides, TRAIL R1, TRAIL R2, Apoptosis inhibitor 2, FasL, Exisulind;
molecules which hamper cell growth such as 2-Deoxy-D-glucose, oligomycin, or Rapamycin or Rapamycin analogues.

62. The composition of claim 60 wherein the chemotherapeutic agent is Temozolomide.

63. The composition of claim 60 wherein the chemotherapeutic agent is 2-Deoxy-D-glucose.

64. The composition of claim 60 wherein the chemotherapeutic agent is Apoptosis inhibitor 2.

65. The composition of claim 60 wherein the chemotherapeutic agent is rapamycin.

66. A method of manufacturing a medicament for treating a patient with cancer, the method comprising utilizing a composition of claim 51.

67. A method of manufacturing a medicament for treating a patient with glioma, the method comprising utilizing a composition of claim 51.

68. A method of identifying an antagonist to cell growth and cell cycle progression from G₁ to S phase comprising the steps of:
a. contacting a cell culture with a sample compound;
b. detecting the concentration of epithelial cell transforming sequence 2 (Ect2) and protein expression of p27^{kip1} in the cell; and
c. detecting the concentration of Ect2 and the protein expression of p27^{kip1} in a second cell culture not contacted with the sample compound,
whereby a decrease in the Ect2 concentration and an increase in protein expression of p27^{kip1} within the cell culture contacted with the sample compound in relation to the second cell culture indicates the sample compound is an antagonist.

69. The method of claim 68 wherein the first and second cell culture are human glioma cells.
